(19) United States
(12) Patent Application Publication
(10) Pub. No.: US 2026/0108393 A1
(43) Pub. Date: Apr. 23, 2026

(54) FRAGMENTATION PATTERNS TO FACILITATE REMOVAL OF A CRYSTALLINE LENS

(71) Applicant: ALCON INC., Fribourg (CH)

(72) Inventors: Keith Watanabe, Irvine, CA (US); Victor Duran Melgoza, Fort Worth, TX (US); Sukit Lertsuntivit, Lake Forest, CA (US); Abraham Carrera Torralvo, Barcelona (ES); Richard Ferdon, Lake Forest, CA (US); Jamshid Ourmazdi, Irvine, CA (US)

(21) Appl. No.: 19/352,599

(22) Filed: Oct. 8, 2025

Related U.S. Application Data
(60) Provisional application No. 63/708,315, filed on Oct. 17, 2024.

(51) Int. Cl. A61F 9/008

(52) U.S. Cl. CPC A61F 9/00825; A61F 2009/00844; A61F 2009/0087; A61F 2009/00897

(57) ABSTRACT

In one or more embodiments, a system configured to create a fragmentation pattern in the crystalline lens of an eye includes a laser system and a computer system. The laser system directs a laser beam towards the crystalline lens. The laser system has a z-axis and xy-planes orthogonal to the z-axis. The computer system instructs the laser system to direct the laser beam toward the crystalline lens to create a star pattern to segment the crystalline lens. The star pattern at one or more xy-planes has includes a star. The star comprises an isotoxal star with a central polygon and acute vertices. The central polygon has three or more sides. Each acute vertex forms a triangular area with a side of the central polygon to yield a plurality of triangular areas.

### FIELD

Embodiments of the present disclosure relate to fragmentation patterns used to facilitate removal of a crystalline lens.

### BACKGROUND

A cataract is a clouding of the natural crystalline lens of the eye. Cataract surgery involves removing the cloudy crystalline lens from the eye and may involve inserting an artificial intraocular lens (IOL) to replace the crystalline lens. Phacoemulsification may be used to emulsify the crystalline lens to allow an aspirator to remove the emulsified lens.

### SUMMARY

In one or more embodiments, a system configured to create a fragmentation pattern in the crystalline lens of an eye includes a laser system and a computer system. The laser system directs a laser beam towards the crystalline lens. The laser system has a z-axis and xy-planes orthogonal to the z-axis. The computer system instructs the laser system to direct the laser beam toward the crystalline lens to create a star pattern to segment the crystalline lens. The star pattern at one or more xy-planes has includes a star. The star comprises an isotoxal star with a central polygon and acute vertices. The central polygon has three or more sides. Each acute vertex forms a triangular area with a side of the central polygon to yield a plurality of triangular areas.

One or more embodiments may include none, one, some, or all of the following features:
The central polygon comprises parallel central region lines.
The central polygon comprises first parallel central region lines and second parallel central region lines, where the second parallel central region lines are substantially orthogonal to the first parallel central region lines.
The star pattern comprises one or more chop line(s). A chop line may intersect within the central polygon. A chop line may bisect the central polygon.
The star pattern comprises a first chop line and a second chop line, where the first chop line is substantially orthogonal to the second chop line.
The star pattern comprises a star pattern cross-section disposed at a plane that bisects the star pattern. The star pattern cross-section has a convex anterior curve and a convex posterior curve.
A triangular area has one or more parallel triangular area line(s).
A triangular area has first parallel triangular area lines and second parallel triangular area lines. The second parallel triangular area lines are substantially orthogonal to the first parallel triangular area lines.
The computer system displays a graphical user interface (GUI) that includes one or more pattern selector(s) configured to receive one or more pattern selection(s), and the computer system generates the star pattern according to the pattern selection(s).
The laser system directs the laser beam towards the crystalline lens to form photodisruption sites in the crystalline lens to create the star pattern.
The central polygon includes a generally square shape.

In one or more embodiments, a system configured to create a fragmentation pattern in the crystalline lens of an eye includes a laser system and a computer system. The laser system directs a laser beam towards the crystalline lens. The laser system has a z-axis and xy-planes orthogonal to the z-axis. The computer system instructs the laser system to direct the laser beam toward the crystalline lens to create a cross pattern to segment the crystalline lens. The cross pattern at one or more xy-planes includes a cross with a central region. The cross includes bars, where each bar has a rectangular shape with a longer side and a shorter side. The bars comprise a first bar and a second bar, where a first length of the longer side of the first bar is substantially equivalent to a second length of the longer side of the second bar. The second bar intersects the first bar at the central region, and the second bar is substantially orthogonal to the first bar.

One or more embodiments may include none, one, some, or all of the following features:
At least one bar includes longer lines substantially parallel to the longer side of the bar.
At least one bar includes shorter lines substantially parallel to the shorter side of the bar.
The cross pattern includes one or more chop lines. A chop line may or may not intersect the central region. A chop line may bisect an angle formed by a first bar axis of the first bar and a second bar axis of the second bar.
The cross pattern includes a first chop line and a second chop line. The first chop line bisects a first angle formed by a first bar axis of the first bar and a second bar axis of the second bar, and the second chop line bisects a second angle formed by the first bar axis of the first bar and the second bar axis of the second bar, where the second angle is distinct from the first angle.
The cross pattern has a cross pattern cross-section disposed at a plane that bisects the cross pattern. The cross pattern cross-section has a convex anterior curve and a convex posterior curve.
The computer system displays a graphical user interface (GUI) that has one or more pattern selector(s) that can receive one or more pattern selection(s), and the computer system generates the cross pattern according to the pattern selection(s).
The laser system directs the laser beam towards the crystalline lens to form photodisruption sites in the crystalline lens to create the cross pattern.

### BRIEF DESCRIPTION OF THE DRAWINGS

FIG. 1 illustrates an example of a system that can create a fragmentation pattern in the crystalline lens of an eye, according to at least one embodiment described in the present disclosure;

FIG. 2 illustrates an example of a computing system, according to at least one embodiment described in the present disclosure;

FIGS. 3A through 3C illustrate an example of a cross pattern, according to at least one embodiment described in the present disclosure;

FIGS. 4A and 4B illustrate examples of cross patterns, according to at least one embodiment described in the present disclosure;

FIGS. 5A through 5C illustrate an example of a star pattern, according to at least one embodiment described in the present disclosure;

FIGS. 6A through 6E illustrate examples of star patterns, according to at least one embodiment described in the present disclosure;

FIG. 7 illustrates an example of a cross pattern graphical user interface (GUI) that may be used to select the features of a cross pattern, according to at least one embodiment described in the present disclosure;

FIG. 8 illustrates an example of a star pattern graphical user interface (GUI) that may be used to select the features of a star pattern, according to at least one embodiment described in the present disclosure; and

FIG. 9 illustrates an example of a method 900 that may be used to create a fragmentation pattern in the lens of an eye, according to at least one embodiment described in the present disclosure.

### DESCRIPTION OF EXAMPLE EMBODIMENTS

Referring now to the description and drawings, one or more example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

The present disclosure relates to facilitating the removal of the crystalline lens during cataract surgery. The present disclosure describes utilizing a laser beam to segment the crystalline lens. For example, a femtosecond laser beam may be used to create a fragmentation pattern in the crystalline lens to segment the lens into smaller pieces. Phacoemulsification may be used to emulsify the smaller pieces.

One or more embodiments of the present disclosure may provide improvements over previous iterations. For example, segmenting the crystalline lens into smaller pieces according to the embodiments may allow phacoemulsification to more effectively and/or more efficiently emulsify the smaller pieces. As another example, a fragmentation pattern of the embodiments may more effectively and/or efficiently segment the crystalline lens than known fragmentation patterns. As yet another example, embodiments may include a graphical user interface (GUI) that may allow a user to select and/or customize a fragmentation pattern.

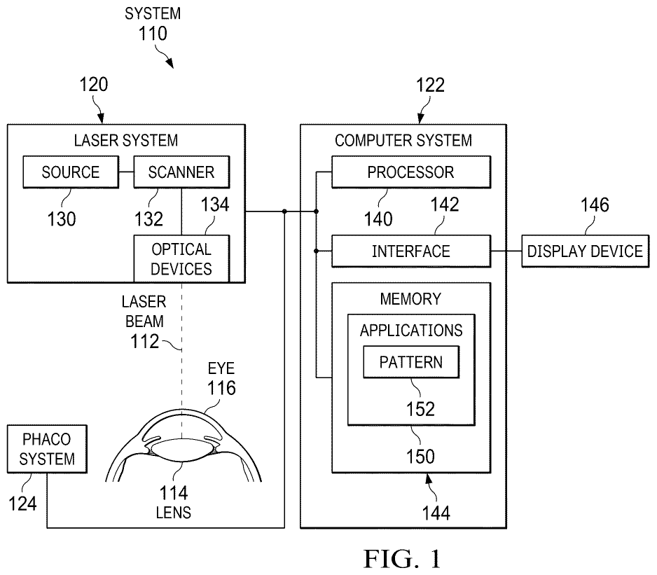

FIG. 1 illustrates an example of a system 110 that can control a laser beam 112 to create a fragmentation pattern in the crystalline lens 114 of an eye 116, according to at least one embodiment described in the present disclosure. In the example, the system 110 includes a laser system 120, a computer system 122, and/or a phacoemulsification (phaco) system 124, which may be coupled as shown. The laser system 120 includes a laser source 130, a scanner 132, and/or optical devices 134, which may be coupled as shown. The computer system 122 includes a processor 140, an interface 142 (which may include a display device 146), and/or a memory 144, which may be coupled as shown. The memory 144 stores applications 150, such as a pattern application 152.

According to an example of operation, the computer system 122 instructs the laser system to direct the laser beam 112 toward the crystalline lens 114 to create a fragmentation pattern in the crystalline lens 114 that segments the crystalline lens 114. The fragmentation pattern may be, e.g., a cross pattern or a star pattern. In an example, the cross pattern includes an equal-armed cross comprising a first bar and a second bar. The second bar may intersect the first bar at the central region and may be substantially orthogonal to the first bar. In another example, the star pattern comprises an isotoxal star with a central polygon and acute vertices. Each acute vertex may form a triangular area with one side corresponding to a side of the central polygon.

For ease of explanation, the embodiments are described using the following example xyz-coordinate system, which may be regarded as the coordinate system of the system 110, although any suitable coordinate system may be used. In the example, the z-axis is aligned with the optical axis of the laser system 120, and an xy-plane is orthogonal to the z-axis. In addition, for ease of explanation, a first dimension may be “substantially equivalent” to a second dimension if the first dimension is within a five percentage error of the second dimension.

The System. The system 110 may be any suitable medical device that performs a medical procedure, such as a surgical and/or diagnostic procedure. In one or more embodiments, the system 110 may be a surgical system that performs cataract surgery (e.g., femtosecond laser assisted cataract (FLAC) surgery) to remove a crystalline lens from the eye. In FLAC surgery, a laser (e.g. a femtosecond laser) may be used to create photodisruptions in the crystalline lens arranged in a fragmentation pattern to segment and/or soften the crystalline lens, in order to facilitate removal of the crystalline lens.

Laser System. The laser system 120 directs the laser beam 112 towards the crystalline lens 114 of the eye 116 to segment and/or soften the crystalline lens 114. For example, the laser source 130 generates the laser beam 112, and the scanner 132 guides the laser beam 112. One or more optical devices 134 direct the laser beam 112 towards the crystalline lens 114 of the eye 116. The laser source 130 may be a femtosecond laser or other ultrashort pulse laser. The laser beam 112 may have any suitable pulse duration, such as in the order of nanoseconds, picoseconds, femtoseconds, or attoseconds. The laser beam 118 may have any suitable wavelength, such as in the range of 150 nanometers (nm) to 20 micrometers (μm). Examples of ranges include the ultraviolet (e.g., in the range of 180 to 400 nm, such as 190 to 195 nm and/or 345 to 355 nm), visible, or infrared wavelength (e.g., in the range of 1050 to 1250 and/or 1250 to 1500 nm). The laser beam 112 may process the material of the crystalline lens 114 in any suitable manner, e.g., photodisrupt the material.

Computer System. The computer system 122 performs operations to segment and/or soften the crystalline lens 114, including sending instructions to other components of the system 110 (e.g., the laser system 120) to perform operations (e.g., direct the laser beam 112 towards the eye 116). The computer system 122 may use applications 150 to perform the operations. For example, the pattern application 152 may be used to instruct the laser system 120 to create a fragmentation pattern in the crystalline lens 114 in the eye 116. Examples of fragmentation patterns include a cross pattern and a star pattern, which are described in more detail herein. In one or more embodiments, the pattern application 152 may be used to display a graphic user interface (GUI) that includes pattern selectors that a user may use to select a pattern and/or customize features of the pattern. Examples of GUIs are described in more detail herein.

Phacoemulsification (Phaco) System. The phacoemulsification (phaco) system 124 removes the lens 114 from the eye 116. In one or more embodiments, the phaco system 124 uses ultrasonic energy to emulsify the fragmented lens 114 and then suctions the lens 114 out of the eye 116. In other embodiments, the phaco system 124 may remove the fragmented lens 114 from the eye 116 without the use of ultrasonic energy. In some embodiments, the fragmentation pattern may include features that soften the crystalline lens such that a lower amount of ultrasonic energy is utilized to remove the crystalline lens as compared to a lens that has not been softened.

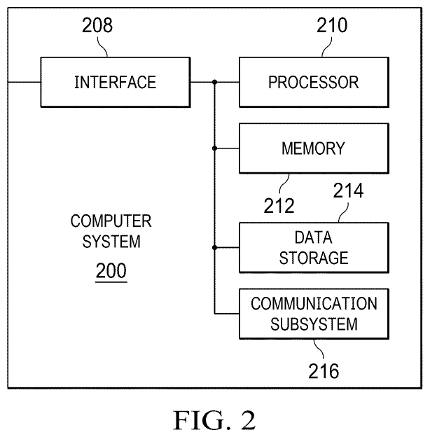

FIG. 2 illustrates an example of a computing system 200, according to at least one embodiment described in the present disclosure. The computing system 200 may include an interface 208, a processor 210, a memory 212, a data storage 214, and/or a communication subsystem 216, any or all of which may be communicatively coupled. Any or all of the computing system 200 may be implemented as computer hardware and/or software. One or more components of a computer system described herein may be implemented as described with reference to the computing system 200.

In the example, the interface 208 may receive input to the computing system 200 and/or send output from the computing system 200, and may be used to exchange information between, e.g., software, hardware, one or more peripheral devices, one or more users, and/or any suitable combinations of any of the preceding. A user interface is a type of interface that a user can utilize to communicate with (e.g., send input to and/or receive output from) the computing system 200. Examples of user interfaces include displays, Graphical User Interfaces (GUIs), touchscreens, foot pedals, keyboards, computer mouses (or mice), gesture sensors, microphones, and speakers.

Generally, the processor 210 may include any suitable special-purpose or general-purpose computer, computing entity, or processing device including various computer hardware or software modules and may be configured to execute instructions stored on any applicable computer-readable storage media. For example, the processor 210 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. Although illustrated as a single processor in FIG. 2, the processor 210 may include any number of processors distributed across any number of network or physical locations that are configured to perform individually or collectively any number of operations described in the present disclosure.

The processor 210 may perform any suitable operations. In some embodiments, the processor 210 may interpret and/or execute program instructions and/or process data stored in the memory 212, the data storage 24, or the memory 212 and the data storage 214. In some embodiments, the processor 210 may fetch program instructions from the data storage 214 and load the program instructions into the memory 212. After the program instructions are loaded into the memory 212, the processor 210 may execute the program instructions, such as instructions to perform any of the methods disclosed herein, respectively.

The memory 212 and the data storage 214 may include computer-readable storage media or one or more computer-readable storage mediums for carrying or having computer-executable instructions or data structures stored thereon. Such computer-readable storage media may be any available media that may be accessed by a general-purpose or special-purpose computer, such as the processor 210.

By way of example, and not limitation, such computer-readable storage media may include non-transitory computer-readable storage media including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 210 to perform a certain operation or group of operations.

The communication subsystem 216 may include any component, device, system, or combination thereof that is configured to transmit, receive, and/or otherwise exchange information over a network in order to communicate with any suitable entity, such as with other devices at other locations or at the same location or even within the same system. The communication subsystem 216 may provide for communication among the devices described in the present disclosure, communication networks, computing devices, and other systems. For example, the communication subsystem 216 may allow the system 200 to communicate with other systems, such as other computing devices and/or networks. In some embodiments, the communication subsystem 216 may include a modem, a network card (wireless or wired), an optical communication device, an infrared communication device, a wireless communication device (such as an antenna), and/or chipset. Examples of communication subsystem 216 include a Bluetooth device, an 802.6 device (e.g., that can communicate with a Metropolitan Area Network (MAN)), a WiFi device, a WiMax device, cellular communication facilities, and/or the like.

One skilled in the art will recognize that modifications, additions, or omissions may be made to the system 200 without departing from the scope of the present disclosure. For example, the system 200 may include more or fewer components than those explicitly illustrated and described.

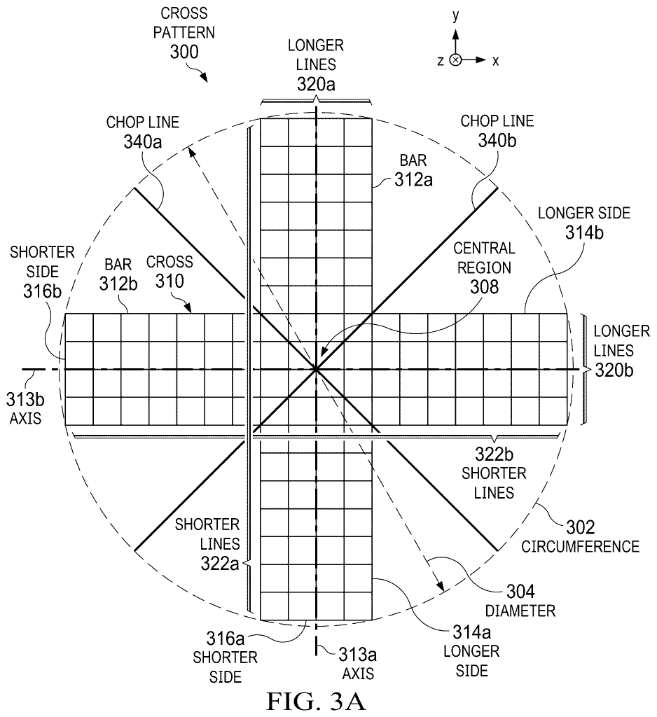

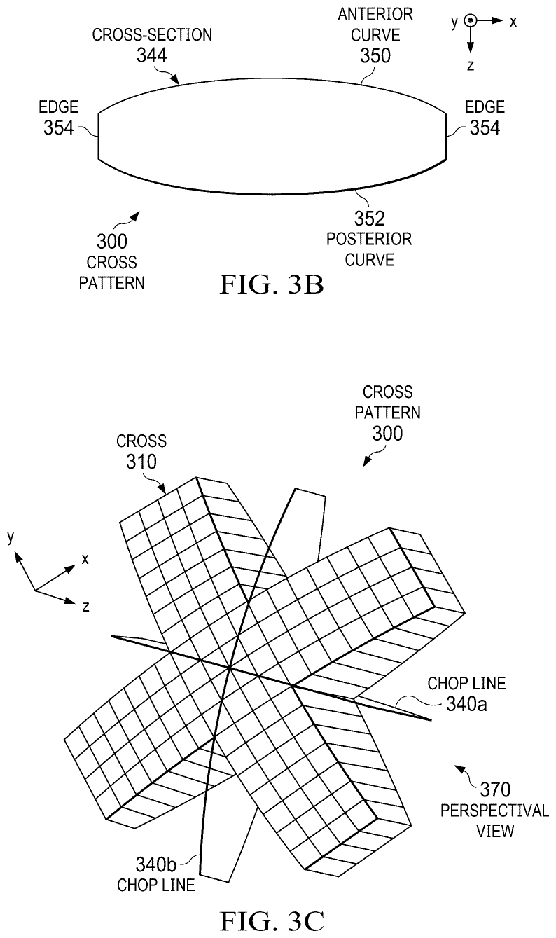

FIGS. 3A through 6E illustrate examples of fragmentation patterns, according to at least one embodiment described in the present disclosure. A fragmentation pattern may have any suitable size and shape, as described herein. For example, a fragmentation pattern may have a diameter (as described herein) that is less than or equal to the diameter of the crystalline lens, such as a diameter in the range of 0 to 0.5, 0.5 to 1, and/or 1 to 1.5 millimeters less than or equal to the diameter of the crystalline lens. As another example, a fragmentation pattern may have a thickness (e.g., a distance between an anterior curve and a posterior curve, as described herein) that is less than or equal to the thickness of the crystalline lens, such as a thickness in the range of 0 to 0.5, 0.5 to 1, and/or 1 to 1.5 millimeters less than or equal to the thickness of the crystalline lens.

FIGS. 3A through 3C illustrate an example of a cross pattern 300, according to at least one embodiment described in the present disclosure. FIG. 3A shows a layer of the cross pattern 300 in an xy-plane, FIG. 3B shows a cross-section 344 of the cross pattern 300 in an xz-plane, and FIG. 3C shows a perspectival view 370 of the cross pattern 300.

Turning to FIG. 3A, the cross pattern 300 includes a cross 310 and may include one or more chop lines 340 (340a and/or 340b). The cross 310 has a central region 308, e.g., the centroid of the cross 310. The outer edges of the cross 310 may be used to designate a circumference 302 of the cross pattern 300, which in turn may be used to designate a diameter 304 of the cross pattern 300.

The cross 310 includes bars 312 (312a and 312b). Each bar 312 (312a or 312b) has an axis 313 (313a or 313b, respectively), which may longitudinally bisect the respective bar 312 (312a or 312b, respectively). The bars 312 (312a and 312b) may be substantially the same length or the bars 312 (312a and 312b) may be different lengths. The bars 312 (312a and 312b) may intersect one another at any suitable point and at any suitable angle. In the example, the bars 312 (312a and 312b) intersect each other at the midpoints of their respective axis 313 (313a and 313b, respectively), and the axis 313a is orthogonal to the axis 313b.

Each bar 312 (312a or 312b) has a generally rectangular shape with a longer side 314 (314a or 314b, respectively) and a shorter side 316 (316a or 316b, respectively). Each bar 312 (312a or 312b) has any suitable number of longer lines 320 (320a or 320b, respectively) substantially parallel to the longer side 314 (314a or 314b, respectively) of the bar 312 (312a or 312b, respectively). In the example, the bars 312 (312a and 312b) each have five longer lines 320 (320a and 320b, respectively), but may have any suitable number of longer lines 320 (320a and 320b, respectively), e.g., 2 to 5, 5 to 10, and/or 10 to 15 longer lines 320 (320a and 320b, respectively). Moreover, the bars 312 (312a and 312b) may have the same number of longer lines 320 (320a and 320b, respectively) or may each have a different number of longer lines 320 (320a and 320b, respectively).

Each bar 312 (312a or 312b) has any suitable number of shorter lines 322 (322a or 322b, respectively) substantially parallel to the shorter side 316 (316a or 316b, respectively) of the bar 312 (312a or 312b, respectively). In the example, the bars 312 (312a and 312b) each have 19 shorter lines 322 (322a and 322b, respectively), but may have any suitable number of shorter lines 322 (322a and 322b, respectively), e.g., 2 to 10, 10 to 20, 20 to 30, and/or 30 to 40 shorter lines 322 (322a and 322b, respectively). Moreover, the bars 312 (312a and 312b) may have the same number of shorter lines 322 (322a and 322b, respectively) or may have a different number of shorter lines 322 (322a and 322b, respectively). In one or more embodiments, the longer lines 320 and shorter lines 322 may form a grid pattern.

In one or more embodiments, the cross pattern 300 may include one or more chop lines 340 (340a and/or 340b). In general, a chop line may pass through the cross 310 or may bypass the cross 310. For example, a chop line may start from the outside of the cross 310, pass through the cross 310, and continue to the other side of the cross 310. As another example, a chop line may start from the outside of the cross 310, stop within the cross 310, and then the chop line may continue on the other side of the cross 310. In the illustrated example, each chop line 340 (340a or 340b) passes through the cross 310.

The chop lines 340 (340a and 340b) may be positioned relative to the cross 310 in any suitable manner. In the example, the respective midpoints of the chop lines 340 (340a and 340b) are located substantially at the central region 308 of the cross 310. The chop lines 340 (340a and 340b) may intersect one another at any suitable point and at any suitable angle. In the example, the chop lines 340 (340a and 340b) intersect each other at their respective midpoints, and the chop line 340a is orthogonal to the chop line 340b. In the example, the chop line 340a bisects the angle formed by the axis 313a and the axis 313b in the-x and +y directions, and the chop line 340b bisects the angle formed by the axis 313a and the axis 313b in the +x and +y directions.

Turning to FIG. 3B, the cross-section 344 of the cross pattern 300 may be taken at any suitable xz-plane, such as the plane along the axis 313b of the bar 312b. The cross-section 344 shows an anterior curve 350, a posterior curve 352, and an edge 354. The anterior curve 350 and the posterior curve 352 may have any suitable shape. In the example, the anterior curve 350 and the posterior curve 352 are convex, i.e., curved outwardly, relative to a center of the cross pattern 300 but may have any suitable shape. The edge 354 may have any suitable shape. In the example, the edge 354 is straight and parallel to the z-axis. In other embodiments, the edge 354 may be curved, e.g., to complete the ellipse formed by the anterior curve 350 and the posterior curve 352.

Turning to FIG. 3C, FIG. 3C shows a perspectival view 370 of the cross pattern 300. The perspectival view 370 shows the cross 310 and chop lines 340 (340a and 340b).

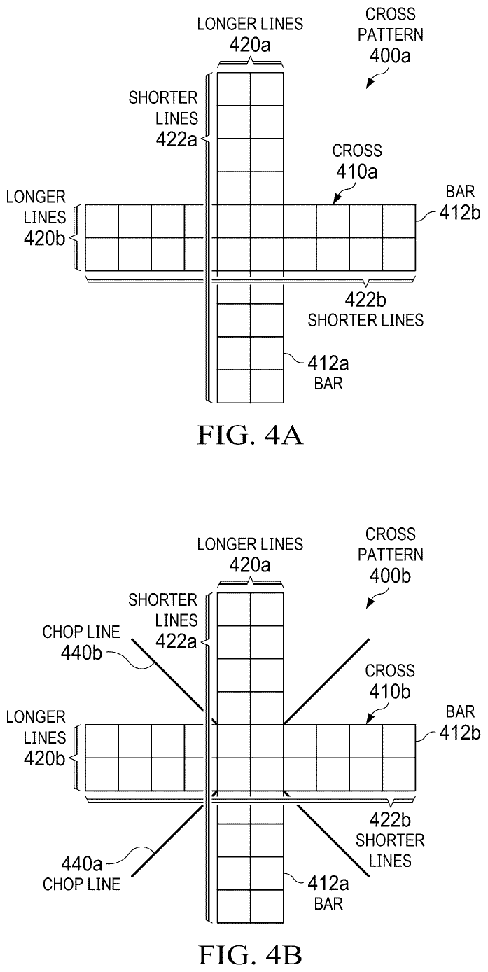

FIGS. 4A and 4B illustrate examples of cross patterns 400 (400a and 400b), according to at least one embodiment described in the present disclosure.

Turning to FIG. 4A, the cross pattern 400a has a cross 410a with bars 412 (412a and 412b). In the example, the bars 412 (412a and 412b) each have three longer lines 420 (420a and 420b, respectively) and 11 shorter lines 422 (422a and 422b, respectively).

Turning to FIG. 4B, the cross pattern 400b has a cross 410b that is substantially similar to the cross 410a of FIG. 4A. The cross pattern 400b has chop lines 440 (440a and 440b). In the example, each chop line 440 (440a and 440b) stops when the chop line 440 (440a and 440b) reaches the cross 410a and continues on the other side of the cross 410b, i.e., each chop line 440 bypasses the cross 410b.

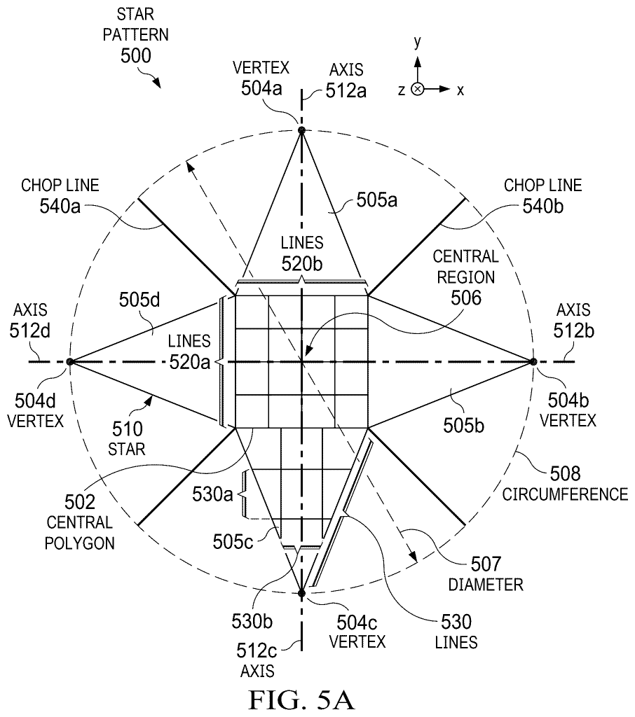

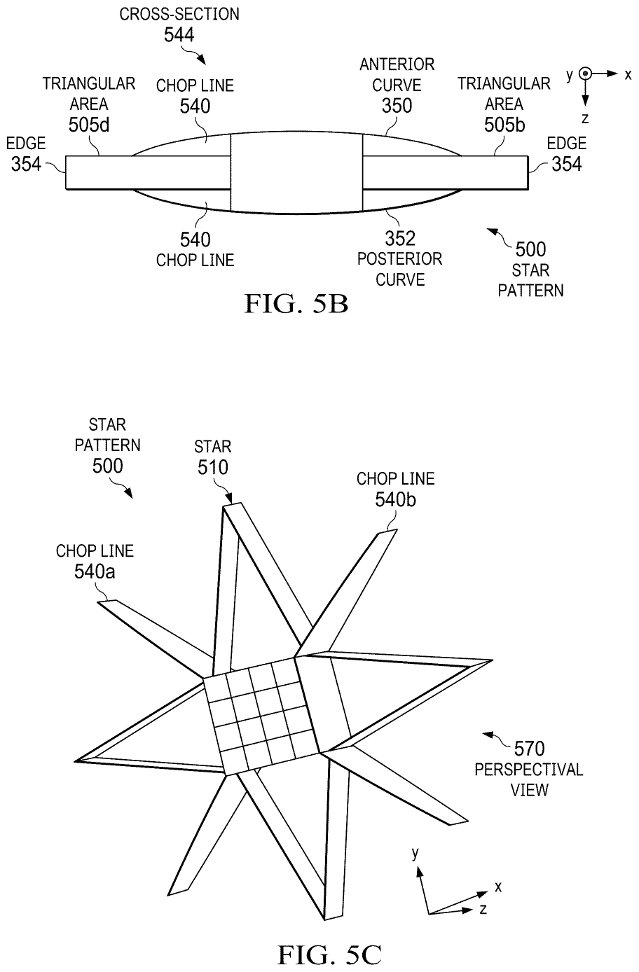

FIGS. 5A through 5C illustrate an example of a star pattern 500, according to at least one embodiment described in the present disclosure. FIG. 5A shows a layer of the star pattern 500 in an xy-plane, FIG. 5B shows a cross-section 544 of the star pattern 500 in an xz-plane, and FIG. 5C shows a perspectival view 570 of the star pattern 500.

Turning to FIG. 5A, the star pattern 500 includes a star 510 comprising an isotoxal star. The star 510 has a central polygon 502 (e.g., a regular polygon) and any suitable number of acute vertices 504 (e.g., 504a, 504b, 504c, and/or 504d). For example, a star 510 with a central polygon 502 with a number N sides may have the number N acute vertices 504. In the example, the star 510 has a central polygon 502 with four sides, and the star 510 also has four vertices 504 (504a, 504b, 504c, and 504d). Each vertex 504 (504a, 504b, 504c, or 504d) forms a triangular area 505 (505a, 505b, 505c, or 505d, respectively) with a side of the central polygon 502. The central polygon 502 has a central region 506, e.g., the centroid of the central polygon 502. The vertices 504 (504a, 504b, 504c, and/or 504c) may be used to designate a circumference 508 of the star pattern 500, which in turn may be used to designate a diameter 507 of the star pattern 500. The vertices 504 (504a, 504b, 504c, and/or 504d) and the central region 506 may be used to designate the axes 512 (512a, 512b, 512c, and/or 512d, respectively) of the star 510.

The central polygon 502 includes any suitable number of central region lines 520 (520a and/or 520b) that may facilitate segmentation of the crystalline lens. In one or more embodiments, the central region lines 520 may be parallel to one or more sides of the central polygon 502. In the embodiments, the central polygon 502 includes a first set of parallel central region lines 520a and a second set of parallel central regions lines 520a. The first set of parallel central region lines 520a is parallel to a first side of the central polygon 502, and the second set of central regions lines 520a is parallel to a second side of the central polygon 502, where the second side is orthogonal to the first side. In the example, the first set of parallel central region lines 520a includes five lines, and the second set of parallel central regions lines 520a includes five lines, but the sets may include any suitable number of lines, e.g., 2 to 5, 5 to 10, 10 to 15, 15 to 20, 20 to 25, and/or 25 to 30 lines. Moreover, the first set of parallel central region lines 520a and the second set of parallel central regions lines 520a may have substantially the same number of lines, or the first set of parallel central region lines 520a and the second set of parallel central regions lines 520a may have a different number of lines. In one or more embodiments, the first set of parallel central region lines 520a and the second set of parallel central regions lines 520a may form a grid pattern.

In one or more embodiments, one or more triangular areas 505 (505a, 505b, 505c, and/or 505d) may include any suitable number of triangular area lines 530 (530a and/or 530b) that may facilitate segmentation of the crystalline lens. In one or more embodiments, the triangular area lines 530 (530a and/or 530b) may be parallel to one or more sides of the central polygon 502. For example, a triangular area may include a first set of triangular area lines 530a and a second set of triangular area lines 530b. The first set of triangular area lines 530a may be parallel to a first side of the central polygon 502, and the second set of triangular area lines 530b may be parallel to a second side of the central polygon 502, where the second side is orthogonal to the first side. In the example, the first set of triangular area lines 530a includes two lines, and the second set of triangular area lines 530b includes two lines, but the sets may include any suitable number of lines, e.g., 0 to 5, 5 to 10, 10 to 15, 15 to 20, 20 to 25, and/or 25 to 30 lines. Moreover, the first set of triangular area lines 530a and the second set of triangular area lines 530b may have substantially the same number of lines, or the first set of triangular area lines 530a and the second set of triangular area lines 530b may have a different number of lines.

In one or more embodiments, the star pattern 500 may include one or more chop lines 540 (540a and/or 540b). In general, a chop line may pass through the star 510 or may bypass the star 510. For example, a chop line may start from the outside of the star 510, pass through the star 510, and continue to the other side of the star 510. As another example, a chop line may start from the outside of the star 510, bypass the star 510, and continue on the other side of the star 510. In the example, each chop line 540 (540a or 540b) bypasses the star 510.

The chop lines 540 (540a and 540b) may be positioned relative to the star 510 in any suitable manner. In the example, the respective midpoints of the chop lines 540 (540a and 540b) are located substantially at the central region 506 of the star 510. In one or more embodiments, the chop lines 540 (540a and 540b) may intersect one another at any suitable point and at any suitable angle. In the example, the chop lines 540 (540a and 540b) intersect each other at their respective midpoints, and the chop line 540a is orthogonal to the chop line 540b such that the chop lines 540 (540a and 540b) each bisect the central polygon 502. In the example, the chop line 540a bisects the angle formed by the axis 512a and the axis 512d, and the chop line 340b bisects the angle formed by the axis 512a and the axis 512b.

Turning to FIG. 5B, the cross-section 544 of the star pattern 500 may be taken at any suitable xz-plane, such as the plane along the axis 512b of triangular area 505b. The cross-section 544 shows an anterior curve 550, a posterior curve 552, and an edge 354. The anterior curve 550 is proximate to the anterior surface of the crystalline lens, and posterior curve 552 is proximate to the posterior portion of the crystalline lens. The anterior curve 550 and the posterior curve 552 may have any suitable shape. In the example, the anterior curve 550 and the posterior curve 552 are convex, but may have any suitable shape. In the example, the anterior curve 550 and the posterior curve 552 indicate the anterior outline and the posterior outline, respectively, of the chop lines 540. In the example, the triangular areas 505 (505a, 505b, 505c, and 505d) have an anterior outline and a posterior outline that is relatively constant in the z-direction, as indicted in the cross-section 544 towards the edge 554. The edge 554 may have any suitable shape. In the example, the edge 554 is straight and parallel to the z-axis.

Turning to FIG. 5C, the perspectival view 570 of the star pattern 500 shows the star 510 and chop lines 540 (540a and 540b).

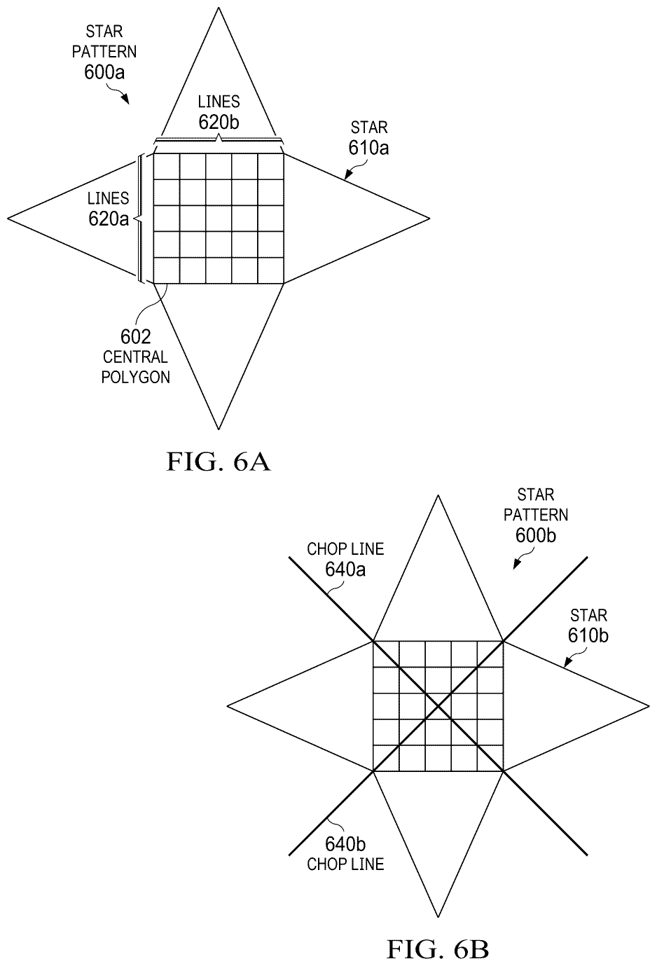

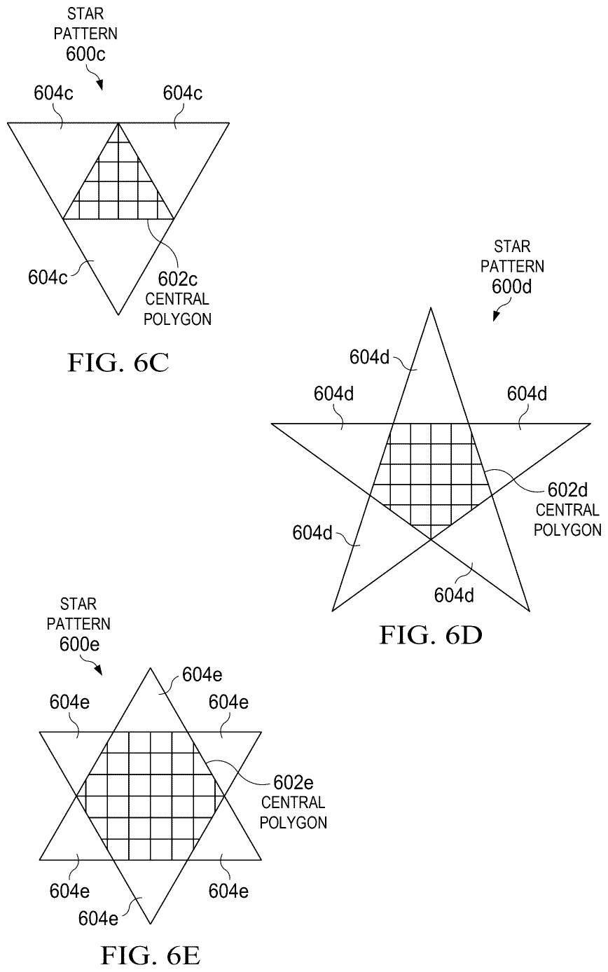

FIGS. 6A through 6E illustrate other examples of star patterns 600 (600a through 600e), according to at least one embodiment described in the present disclosure.

FIG. 6A shows the star pattern 600a. The star pattern 600a has a star 610a with a central polygon 602. In the example, the central polygon 602 has a first set of parallel lines 620a and a second set of parallel lines 620b, where the first set of parallel lines 620a is orthogonal to the second set of parallel lines 620b. In the example, the first set of parallel lines 620a has six lines, and the second set of parallel lines 620b has six lines; however, the first set of parallel lines 620a and the second set of parallel lines 620b may have any suitable number of lines, as described herein.

FIG. 6B shows the star pattern 600b. The star pattern 600b has a star 610b that is substantially similar to the star 610a of FIG. 6A. The star pattern 600b also has chop lines 640 (640a and 640b). Each chop line 640 (640a and 640b) completely intersects with the star 610b.

FIG. 6C shows the star pattern 600c. The star pattern 600c has a three-sided central polygon 602c and three triangular areas 604c.

FIG. 6D shows the star pattern 600d. The star pattern 600d has a five-sided central polygon 602c and five triangular areas 604d.

FIG. 6E shows the star pattern 600e. The star pattern 600e has a six-sided central polygon 602c and six triangular areas 604e.

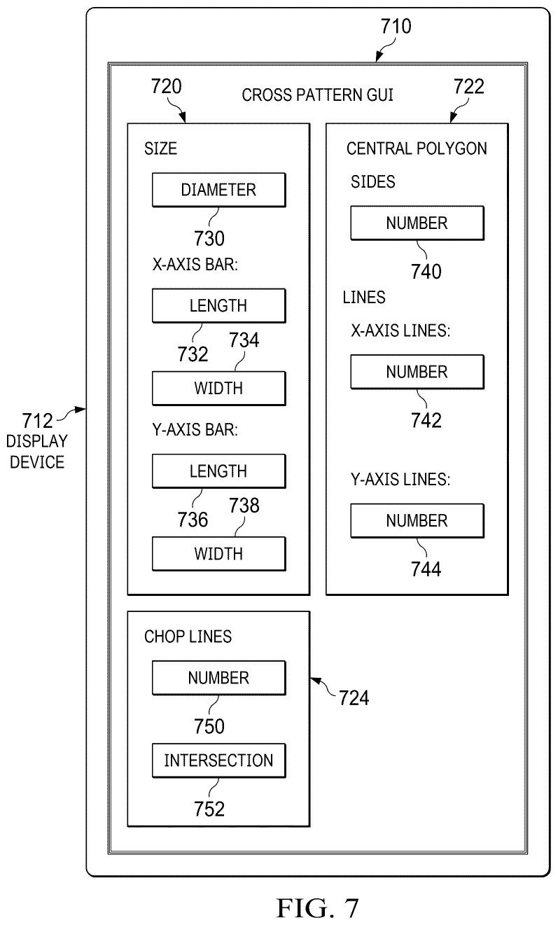

FIG. 7 illustrates an example of a cross pattern graphical user interface (GUI) 710 that may be used to select the features of a cross pattern, according to at least one embodiment described in the present disclosure. The cross pattern GUI 710 may be displayed by a display device 712. In the example, the cross pattern GUI 710 includes one or more size selectors 720, one or more central polygon selectors 722, and/or one or more chop lines selectors 724. The size selectors 720 includes a diameter selector 730, an x-axis bar length selector 732, an x-axis bar width selector 734, a y-axis bar length selector 736, and/or a y-axis bar width selector 738. The central polygon selectors 722 includes a number of sides selector 740, a number of x-axis lines selector 742, and/or a number of y-axis lines selector 744. The chop lines selectors 724 includes a number of chop lines selector 750 and/or a chop line intersection selector 752.

In the example, the diameter selector 730 of the size selectors 720 may be used to select the diameter of the cross pattern, which may be the length of one bar or both bars or a mathematical function (e.g., the average) of the length of the one or more bars. The x-axis bar length selector 732 may be used to select the length (along the x-axis) of the x-axis bar, and the x-axis bar width selector 734 may be used to select the width (along the y-axis) of the x-axis bar. The y-axis bar length selector 736 may be used to select the length (along the y-axis) of the y-axis bar, and the y-axis bar width selector 738 may be used to select the width (along the x-axis) of the y-axis bar.

In the example, the number of x-axis lines selector 742 of the central polygon selectors 722 may be used to select the number of lines parallel to the x-axis in the central polygon. The number of y-axis lines selector 744 may be used to select the number of lines parallel to the y-axis in the central polygon.

In the example, the number of chop lines selector 750 of the chop lines selectors 724 may be used to select the number of chop lines to include in the cross pattern. The chop line intersection selector 752 may be used to select whether a chop line intersects, partially intersects, or does not intersect the central polygon.

In some embodiments, an option may be selectable to maintain symmetry between the x-axis features and the y-axis features, such as the number of lines, the length, the width, etc. In some embodiments, other approaches may be used to modify or customize the cross pattern, such as by line density, or any other setting.

In some embodiments, the cross pattern GUI 710 may be implemented on a touch screen in which the displayed cross pattern may be selectable and adjustable. For example, a surgeon may touch and drag an edge of the cross pattern, and the GUI 710 may cause a corresponding change to the other edges of the cross pattern to maintain a symmetrical cross pattern.

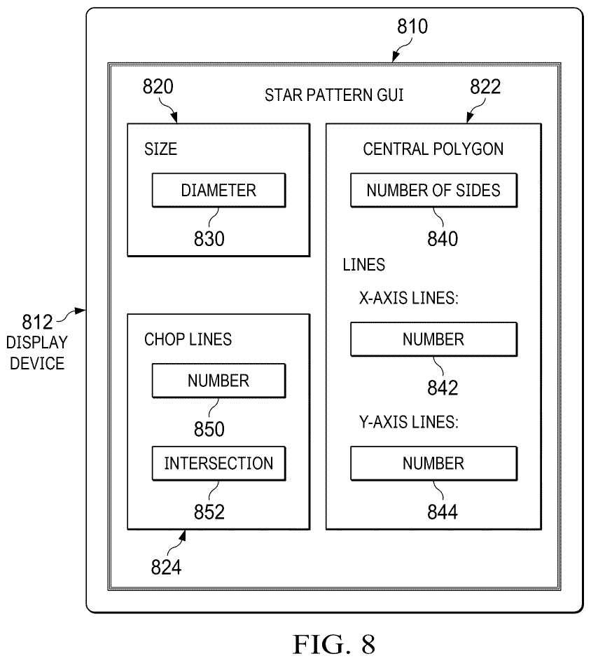

FIG. 8 illustrates an example of a star pattern graphical user interface (GUI) 810 that may be used to select the features of a star pattern, according to at least one embodiment described in the present disclosure. The star pattern GUI 810 may be displayed by a display device 812.

In the example, the star pattern GUI 810 includes one or more size selectors 820, one or more central polygon selectors 822, and/or one or more chop lines selectors 824. The size selectors 820 includes a diameter selector 830. The central polygon selectors 822 includes a number of sides selector 840, a number of x-axis lines selector 842, and/or a number of y-axis lines selector 844. The chop lines selectors 824 includes a number of chop lines selector 850 and/or a chop line intersection selector 852.

In the example, the diameter selector 830 of the size selectors 820 may be used to select the diameter of the star pattern. The number of sides selector 840 of the central polygon selectors 822 may be used to select the number of sides of the central polygon. The number of x-axis lines selector 842 may be used to select the number of lines parallel to the x-axis in the central polygon. The number of y-axis lines selector 844 may be used to select the number of lines parallel to the y-axis in the central polygon.

In the example, the number of chop lines selector 850 of the chop lines selectors 824 may be used to select the number of chop lines to include in the star pattern. The chop line intersection selector 852 may be used to select whether a chop line intersects, partially intersects, or does not intersect the central polygon.

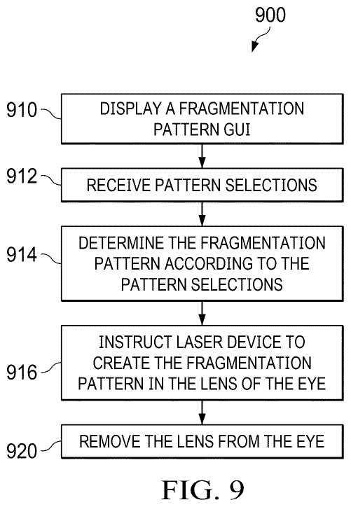

FIG. 9 illustrates an example of a method 900 that may be used to create a fragmentation pattern in the lens of an eye, according to at least one embodiment described in the present disclosure. In the example, one or more blocks of the method 900 may be performed by a computer system, a laser system, and/or a phacoemulsification (phaco) system.

At block 910, a computer system displays a fragmentation pattern GUI (e.g., a cross pattern GUI and/or a star pattern GUI) that may allow a user to select features of the fragmentation pattern. For example, the fragmentation pattern GUI may allow the used to select the size and/or the shape (e.g., a cross or a star) of the fragmentation pattern. As another example, the fragmentation pattern GUI may allow the user to select whether the fragmentation pattern includes a grid pattern of lines and/or one or more chop lines.

At block 912, the computer system receives pattern selections of features of the fragmentation pattern. For example, the computer system may receive a selection of a cross shape of a selected size or a star shape of a selected size. As another example, the computer system may receive a selection the fragmentation pattern includes or does not include a grid pattern of lines. As yet another example, the computer system may receive a selection the fragmentation pattern includes or does not include one or more chop lines.

At block 914, the computer system determines the fragmentation pattern according to the pattern selections. For example, the computer system may identify a stored pattern that matches the pattern selections. As another example, the computer system may generate a pattern according to the pattern selections. In some embodiments, the block 914 may include displaying the fragmentation pattern as an overlay on an image (e.g., an OCT image or a camera image) of the crystalline lens. In these and other embodiments, the computer system may seek confirmation from a user (e.g., a surgeon, nurse, or technician) of the fragmentation pattern as visualized.

At block 916, the computer system instructs the laser system to create the fragmentation pattern in the lens of the eye to fragment the lens.

At block 920, the computer system instructs the phaco system to facilitate removal of the lens from the eye. The phaco system may or may not use ultrasonic energy to facilitate removal of the lens.

The present disclosure (including the specification, claims, and drawings) includes example embodiments that are intended to aid the reader in understanding the invention and concepts contributed by the inventor to furthering the art and to enable any person skilled in the art to make or use the disclosed embodiments. Modifications (e.g., changes, substitutions, additions, omissions, and/or other modifications) to the embodiments will be readily apparent to those skilled in the art. Accordingly, modifications may be made to the embodiments without departing from the essence of the present disclosure.

In certain instances, modifications may be made to the systems disclosed herein, as apparent to those skilled in the art. For example, parts of a system may be integrated or separated, or an operation of a system may be performed by more, fewer, or other parts. In certain instances, modifications may be made to the methods disclosed herein, as apparent to those skilled in the art. For example, the methods may include more, fewer, or other operations. As another example, certain operations may be optional, combined into fewer operations, or expanded into additional operations. As yet another example, certain operations may be performed in any suitable order or simultaneously.

Furthermore, those skilled in the art will recognize that the present disclosure is not intended to be limited to the example embodiments and that the language of the disclosure is to be accorded the widest scope consistent with the present disclosure. Terms (which may include one or more words) that describe inclusion are generally intended as “open” terms in that they generally do not imply exclusion. For example, the term “including” may be interpreted as “including, but not limited to” or “including at least”; the term “having” may be interpreted as “having, but not limited to” or “having at least”; and the term “comprising” may be interpreted as “comprising, but not limited to”or “comprising at least”, etc.

Additionally, if a specific number is intended, such intent will be explicitly recited in the claim. In the absence of the explicit recitation of a specific number, no such intent is present. If a specific number is explicitly recited, such recitation should be interpreted to mean at least the recited number. For example, the bare recitation of “two Xs”, without other modifiers, may mean “at least two Xs” or “two or more Xs”. Moreover, the use of an indefinite article (e.g., “a” or “an”) or definite article (e.g., “the”) to introduce a noun phrase should not be construed to limit the noun phrase to one, but may be interpreted as an open term “at least one” or “one or more”. This holds even when the same claim includes an open term (e.g., “one or more” or “at least one”) and an indefinite or definite article (e.g., “a” or “an” or “the”).

Moreover, a selection from a list of items should be understood to contemplate a selection of any suitable individual item or any suitable combination of items. For example, the general construction “at least one of A, B, and C” or “one or more of A, B, and C” may include A alone; B alone; C alone; A and B together; A and C together; B and C together; and A, B, and C together. Moreover, any disjunctive term presenting two or more alternative items may be understood to contemplate including one of the items, either of the items, or both items. For example, the general construction “A or B” or “A and/or B” may include A alone, B alone, and A and B together. Additionally, the use of the terms “first,” “second,” “third,” etc. are not necessarily used herein to connote a specific order. For example, the terms “first,” “second,” “third,” etc., may be used to distinguish between different elements.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the Applicants to refer to structures known to those skilled in the art and is not intended to invoke 35 U.S.C. § 112(f).

What is claimed:

1. A system configured to create a fragmentation pattern in a crystalline lens of an eye, comprising:
a laser system configured to direct a laser beam towards the crystalline lens of the eye, the laser system having a z-axis and a plurality of xy-planes orthogonal to the z-axis; and
a computer system configured to instruct the laser system to:
direct the laser beam toward the crystalline lens to create a star pattern to segment the crystalline lens, the star pattern at one or more xy-planes of the plurality of xy-planes comprising a star, the star comprising an isotoxal star having a central polygon and a plurality of acute vertices, the central polygon comprising three or more sides, each acute vertex of the plurality of acute vertices forming a triangular area with a respective side of the central polygon to yield a plurality of triangular areas.

2. The system of claim 1, the central polygon comprising a plurality of parallel central region lines.

3. The system of claim 1, the central polygon comprising:
a plurality of first parallel central region lines; and
a plurality of second parallel central region lines, the second parallel central region lines substantially orthogonal to the first parallel central region lines.

4. The system of claim 1, the star pattern comprising one or more chop lines.

5. The system of claim 4, wherein a chop line of the one or more chop lines bypasses the star.

6. The system of claim 4, wherein a chop line of the one or more chop lines passes through the star.

7. The system of claim 1, the star pattern comprising a first chop line and a second chop line, the first chop line substantially orthogonal to the second chop line.

8. The system of claim 1, the star pattern comprising a star pattern cross-section disposed at a plane that bisects the star pattern, the star pattern cross-section comprising a convex anterior curve and a convex posterior curve.

9. The system of claim 1, a triangular area of the plurality of triangular areas having one or more parallel triangular area lines.

10. The system of claim 1, a triangular area of the plurality of triangular areas having:
a first plurality of parallel triangular area lines; and
a second plurality of parallel triangular area lines, the second plurality of parallel triangular area lines substantially orthogonal to the first plurality of parallel triangular area lines.

11. The system of claim 1, the computer system configured to:
display a graphical user interface (GUI), the GUI comprising one or more pattern selectors configured to receive one or more pattern selections; and
generate the star pattern according to the one or more pattern selections.

12. The system of claim 1, the laser system configured to direct the laser beam towards the crystalline lens of the eye to form a plurality of photodisruption sites in the crystalline lens of the eye to create the star pattern.

13. The system of claim 1, wherein the central polygon includes a generally square shape.

14. A system configured to create a fragmentation pattern in a crystalline lens of an eye, comprising:
a laser system configured to direct a laser beam towards the crystalline lens of the eye, the laser system having a z-axis and a plurality of xy-planes orthogonal to the z-axis; and
a computer system configured to instruct the laser system to:
direct the laser beam toward the crystalline lens to create a cross pattern to segment the crystalline lens, the cross pattern at one or more xy-planes of the plurality of xy-planes comprising a cross having a central region, the cross comprising a plurality of bars, each bar of the plurality of bars having a rectangular shape with a longer side and a shorter side, the plurality of bars comprising a first bar and a second bar, a first length of the longer side of the first bar substantially equivalent to a second length of the longer side of the second bar, the second bar intersecting the first bar at substantially the central region, the second bar substantially orthogonal to the first bar.

15. The system of claim 14, at least one bar of the plurality of bars comprising:
a plurality of longer lines substantially parallel to the longer side of the at least one bar.

16. The system of claim 14, at least one bar of the plurality of bars comprising:
a plurality of shorter lines substantially parallel to the shorter side of the at least one bar.

17. The system of claim 14, the cross pattern comprising one or more chop lines.

18. The system of claim 17, wherein a chop line of the one or more chop lines bypasses the cross.

19. The system of claim 17, wherein a chop line of the one or more chop lines passes through the cross.

20. The system of claim 14, the cross pattern comprising a first chop line and a second chop line, the first chop line bisecting a first angle formed by a first bar axis of the first bar and a second bar axis of the second bar, the second chop line bisecting a second angle formed by the first bar axis of the first bar and the second bar axis of the second bar, the second angle distinct from the first angle.

21. The system of claim 14, the cross pattern having a cross pattern cross-section disposed at a plane that bisects the cross pattern, the cross pattern cross-section comprising a convex anterior curve and a convex posterior curve.

22. The system of claim 14, the computer system configured to:
display a graphical user interface (GUI), the GUI comprising one or more pattern selectors configured to receive one or more pattern selections; and
generate the cross pattern according to the one or more pattern selections.

23. The system of claim 14, the laser system configured to direct the laser beam towards the crystalline lens of the eye to form a plurality of photodisruption sites in the crystalline lens of the eye to create the cross pattern.
